(19) United States
(12) Patent Application Publication
(10) Pub. No.: US 2007/0211212 A1
(43) Pub. Date: Sep. 13, 2007

(54) Eye state sensor

(76) Inventor: Percy Bennwik, Nacka (SE)

Correspondence Address: Pharmacia Corporation;Global Patent Department, P.O. Box 1027, St. Louis, MO, 63006, US

(21) Appl. No.: 10/659,815

(22) Filed: Sep. 11, 2003

Related U.S. Application Data
(60) Provisional application No. 60/413,928, filed on Sep. 26, 2002.

(51) Int. Cl. A61B 3/10

(52) U.S. Cl. 351/221; 351/205

(57) ABSTRACT

A method for sensing the state of an eye of a subject comprises measuring light reflected from an ocular surface and comparing the measured light to a reference. A method for treating an eye of a subject comprises sensing the state of the eye as described above, and controlling whether a substance is delivered to the eye whereby the substance is so delivered only when the eye is sensed to be open. A device for sensing the state of an eye comprises a light source that directs light to an ocular surface of a subject, and a sensor for measuring light reflected from the ocular surface. An apparatus for treating an eye of a subject comprises a device for sensing the state of an eye as described above, an applicator for delivering a substance to the eye, and a control system that permits delivery of the substance when the sensing device detects that the eye is open but prevents delivery of the substance when the sensing device detects that the eye is closed.

This application claims priority of U.S. provisional application Ser. No. 60/413,928 filed on Sep. 26, 2002.

### FIELD OF THE INVENTION

The present invention relates to a method and apparatus for detecting the state of an eye, i.e., whether the eye is open or closed. In particular, the invention relates to a method and apparatus for controlling timing of ocular intervention required in diagnosis, prevention or treatment of an ophthalmic condition, disorder or disease.

### BACKGROUND OF THE INVENTION

In ophthalmic medicine it is frequently desired to administer substances directly to, or otherwise treat, an open eye. Success in such administration or treatment, herein generically referred to as ocular intervention, frequently relies upon ensuring that the eye does not close during or at the moment of intervention.

The voluntary or involuntary act of blinking, i.e., rapidly closing and opening the eye, presents particular problems for ocular intervention. The normal blink rate of a human eye is about 12 to about 20 closures per minute, and the average duration of a blink is about 0.25 seconds. The blink rate can increase due to anxiety or stress, or injury or disease of the eye. Moreover, the very act of ocular intervention, for example delivering a substance to the eye, can provoke an involuntary blink response, resulting in all or part of the substance intended for delivery to the eye itself being deposited instead on the outer surface of an eyelid or being caught by eyelashes.

It would therefore be very advantageous to be able to control the precise timing of an ocular intervention such as administration of a medicament or other substance, in such a way that the intervention cannot occur during a blink or other period of eye closure.

### SUMMARY OF THE INVENTION

There is now provided a method for sensing the state of an eye of a subject, the method comprising measuring light reflected from an ocular surface and comparing the measured light to a reference. An “ocular surface” herein is the outermost surface presented to incident light by the eye or its accessory structures and, depending on the state of the eye can be, for example, the corneal surface of the eye itself or the outer surface of an eyelid.

One or more references can be used as comparators in the method. Typically a reference is a stored data point or set derived from measurement of light reflected from an open eye or a closed eye. Preferably the reference relates to measured light reflected from the subject's own eye or eyes.

There is also provided a method for treating an eye of a subject, the method comprising sensing the state of the eye as described above, and controlling whether a substance is delivered to the eye whereby the substance is so delivered only when the eye is sensed to be open.

Also provided is a device for sensing the state of an eye. Such a device comprises a light source that directs light to an ocular surface of a subject, and a sensor for measuring light reflected from the ocular surface. The device can further comprise a standoff to position and orient the sensor and the optional light source at a consistent distance from and angle to the eye.

Further provided is an apparatus for treating an eye of a subject. This apparatus comprises a device for sensing the state of an eye as described above, an applicator for delivering a substance to the eye, and a control system that permits delivery of the substance when the sensing device detects that the eye is open but prevents delivery of the substance when the sensing device detects that the eye is closed.

It is strongly preferred that the sensing and controlling steps of the method for treating an eye, and the sensing device and control system of the apparatus for treating an eye, are configured to permit detection of a blink and lockout of delivery of the substance for at least the duration of the blink. In practice such configuration requires a sampling frequency, i.e., frequency of measurement of reflected light, of at least about 20 Hz, preferably at least about 50 Hz, more preferably at least about 100 Hz.

### BRIEF DESCRIPTION OF THE DRAWINGS

FIG. 1 is a schematic view of a device for sensing the state of an eye.

FIG. 2 is a schematic view of an apparatus for treating an eye, the apparatus incorporating a device for sensing the state of the eye.

FIG. 3 is a graph of eye reflectivity at a sampling frequency of 100 Hz, over a period of 1 second.

FIG. 4 is a schematic drawing in section view of an illustrative blink-avoiding dispenser useful according to the invention for delivering a medicament to an eye.

### DETAILED DESCRIPTION OF THE INVENTION

A first aspect of this invention relates to a method of, and device for, sensing the state of an eye of a subject, i.e., sensing whether the eye is open or closed. The subject is preferably mammalian, most preferably human. According to the method, electromagnetic radiation, preferably of wavelengths from about 400 to about 10⁵ nm, including visible and infrared light (collectively referred to herein as “light”) reflected from an ocular surface is measured with a sensor. The sensor is preferably selected, conditioned, adjusted or tuned to measure intensity of reflected light at a discrete wavelength, or over a narrow or broad range of wavelengths. The source of the light reflected can be ambient, e.g., sunlight or artificial light, but preferably is an artificial source provided as part of the device, such as an incandescent, fluorescent or electroluminescent light source. Thus a preferred method of the invention further comprises projecting light from a light source on to the ocular surface. An especially preferred light source is a light emitting diode (LED). The sensor is preferably tuned to the particular wavelength or range of wavelengths emitted by the light source.

The light measured can be in the visible spectrum (about 400 nm to about 750 nm) or in the infrared spectrum (about 700 nm to about 100 μm) or both. The present inventors have had superior results using red (about 630 nm to about 750 nm) or infrared (about 2.5 μm to about 25 μm) light, but satisfactory results at other light wavelengths can be achieved with appropriately selected sensors.

The present invention is derived in part from a discovery that reflectivity of an open eye is lower than that of a closed eye, and that, surprisingly, the difference in reflectivity between an open and a closed eye is largely independent of eye color. Thus in most subjects, intensity of the light reflected can be used to detect whether the eye is open or closed. According to the present method, the intensity of reflected light from an ocular surface of a subject is compared to at least one reference to determine the state of the eye. The at least one reference can be a standardized value of reflectivity of an eye in either the open or closed state. Optionally two references can be used, representing standardized values of reflectivity of an open eye and a closed eye.

Reflectivity of eyes, in both open and closed states, varies among subjects. For this reason, it may be preferred to select the references to relate to the particular class of subject whose eye state is to be sensed. The inventors have determined, for example, that eye size and shape can be significant factors affecting reflectivity. References can therefore be selected based on age, sex, ethnic group or other easily determined factors. A reference can also be specifically selected for the individual subject. A reference value for reflectivity when the eye is open and/or closed can be measured and used to determine whether a later measurement of reflected light indicates that the eye is open or closed.

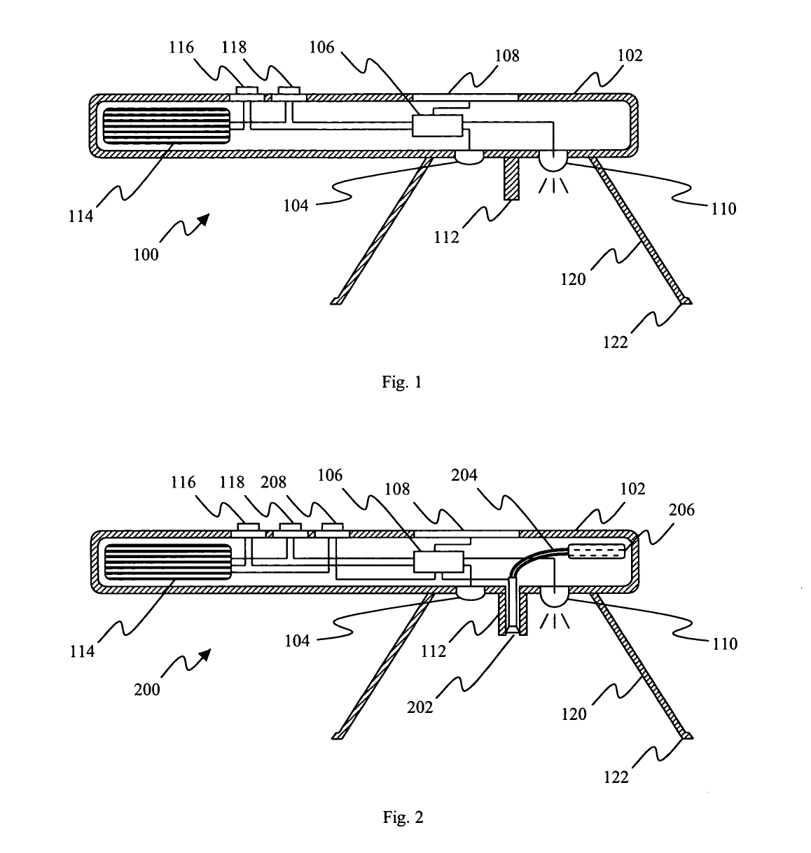

An illustrative device for sensing the state of an eye is shown schematically in FIG. 1. The device 100 comprises a housing 102 having mounted thereon a sensor 104 for measuring intensity of light reflected from a subject's eye. The sensor is connected to a microprocessor 106 which in turn is connected to a display unit, for example a liquid crystal display unit 108. The device 100 as illustrated further comprises a light source, for example an LED 110, mounted near the sensor 104, for directing light to the subject's eye. The sensor 104 is shielded from direct illumination by the light source 110 by means of a shield 112 mounted on the housing 102 and interposed between the light source 110 and the sensor 104. The shield 112 is substantially opaque at least to the wavelength or range of wavelengths of light sensed by the sensor 104. Electrical energy for operation of the device is supplied by any convenient means, external or internal, but in the illustrated embodiment is supplied by a battery 114 removably located within the housing 102.

Operation of the device 100 as illustrated in FIG. 1 is controlled by two actuation means, for example as illustrated, push-button controls. A first push-button 116 can be used to calibrate the device by measuring and storing in a memory unit of the microprocessor 106 a reference value of reflectivity when an eye is open or closed. A second push-button 118 can be used to operate the device to measure reflectivity of an eye and compare it with the stored reference value, and thereby detect whether the eye is opened or closed. Depressing one of the push-buttons 116 or 118 causes the LED 110 to become illuminated and the sensor 104 to sense and measure reflected light.

The device 100 as illustrated in FIG. 1 further comprises a standoff to help consistently position the device relative to the subject's eye such that the light source 110 and the sensor 104 are at a suitable distance from and oriented directly toward the eye. The inventors have found that the position and distance at which the device is held relative to the eye can significantly affect the measurement of reflected light by the sensor. In a preferred embodiment as illustrated, the standoff comprises an eye-cup 120 having a distal rim 122 adapted to contact a surface of the subject's face around the eye to position and orient the sensor 104 and the LED 110 at an appropriate and consistent distance from the eye. The sensor 104, the light source 110 and the shield 112 are all located within a proximal perimeter of the eye-cup 120 defining a locus of attachment of the eye-cup 120 to the housing 102. The eye-cup 120 is preferably designed so that it achieves a substantially consistent spacing from the eye for a majority of subjects. Where, as in the illustrated embodiment, the device has a self-contained light source, the eye-cup 120 is preferably constructed of a material that is substantially opaque to the wavelength or range of wavelengths of light sensed by the sensor 104, so that ambient light does not interfere with measurement of reflected light from the subject's eye.

The sensor 104 can measure the reflected light at a single time point or is preferably programmed via the microprocessor to take measurements of reflected light at a multiplicity of time points over a sampling period. A sampling frequency of about 100 Hz has been found suitable but greater or lesser frequencies can be used if desired. The microprocessor 106 can process a stream of signals received from the sensor 104 and, based on fluctuations in the signals determine when an eye is open and when it is closed. Experimental data using red and infrared wavelengths of light indicate that on average reflectivity of an open eye is about 10% to about 57% lower than that of a closed eye.

The method and device of the invention, as illustrated by device 100 of FIG. 1, are useful in determining the state of an eye for any purpose, but especially as an aid in determining when to treat or not to treat the eye. For example, in the case of administration of a substance to the eye for diagnosis, prevention or treatment of an ophthalmic condition, disease or disorder, the present method and device can permit the substance to be delivered only while the eye is open. In other cases, it may be desirable not to take some action when the eye is open, and the method and device of the invention permit the state of the eye to be monitored to prevent such action when the device senses that the eye is open.

A further embodiment of the invention is shown schematically in FIG. 2. Apparatus 200 in FIG. 2 is similar in construction to device 100 of FIG. 1, and corresponding parts are identified with corresponding reference numerals. However, apparatus 200 further comprises an applicator 202 for a substance, which is fed via a conduit 204 from a reservoir 206. The reservoir 206 can be external to the apparatus but, as illustrated, is preferably contained within the housing 102 of the apparatus, either as a refillable vessel or, most preferably, a replaceable cartridge. An additional actuation means, for example push-button control 208, acts as a trigger for operating the applicator. The microprocessor 106 is programmed to provide a lockout so that the push-button 208 does not actuate the applicator unless the sensor 104 detects that the eye is open. Thus when it is desired to deliver a substance to a subject's eye, the user (the subject or another person) locates the apparatus over the subject's eye, for example using the eye-cup 120 to position and orient the apparatus, and operates the push-button 208. Because of the lockout, the substance is not delivered by the applicator 202 unless the eye is open. As shown in FIG. 2, the applicator 202 or a nozzle thereof can usefully be incorporated into the shield 112, but alternative arrangements are possible.

Any suitable applicator can be used. For example, it can be a spray or droplet generating device as disclosed in any of the patents individually cited below and incorporated herein by reference.

U.S. Pat. No. 4,834,728 to McKenna.

U.S. Pat. No. 5,201,726 to Kirkham.

U.S. Pat. No. 5,578,021 to Cornish.

U.S. Pat. No. 5,588,564 to Hutson & Demangus.

U.S. Pat. No. 5,894,841 to Voges.

U.S. Pat. No. 6,033,389 to Cornish.

The applicator can alternatively be a unit-dose dispenser such as disclosed in the publications individually cited below and incorporated herein by reference.

International Patent Publication No. WO 96/06581.

International Patent Publication No. WO 97/23177.

International Patent Publication No. WO 99/16467.

International Patent Publication No. WO 02/62488.

The applicator can alternatively be an electrodynamic dispenser, such as disclosed in U.S. Pat. No. 4,952,212 to Booth et al., incorporated herein by reference.

The applicator can alternatively be a bubble jet dispenser, such as disclosed in U.S. Pat. No. 5,368,582 to Bertera, incorporated herein by reference.

The applicator can alternatively be an electromechanical or electroacoustic dispenser as disclosed in U.S. Pat. No. 5,518,179 to Humberstone et al., incorporated herein by reference.

The applicator can alternatively be an electromechanical dispenser as disclosed in U.S. Pat. No. 5,838,350 to Newcombe et al., incorporated herein by reference.

While any suitable applicator can be combined with the eye state sensor of the invention to provide an apparatus for treating an eye as described above, it is strongly preferred to use an applicator that is capable of very rapid response to a signal from the eye state sensor. By use of such an applicator, it is possible to negate the effect of involuntary blinking. For example, the apparatus can be programmed to actuate the applicator immediately, e.g., within about 0.5 second, preferably within about 0.25 second, more preferably within about 0.1 second, after completion of a blink, thereby minimizing the probability that another blink will occur during delivery of a substance by the applicator. As another example, the apparatus can be programmed to permit manual actuation of the applicator at any time that the eye state sensor detects an open eye, to interrupt operation of the applicator if a blink occurs, for at least the duration of the blink, and to restart operation of the applicator after the blink if the complete pre-programmed dose of the substance has not yet been delivered.

A preferred class of applicator is an electrically energizable droplet generating device, for example as used in the printing art, most preferably a thermal resistor bubble jet device.

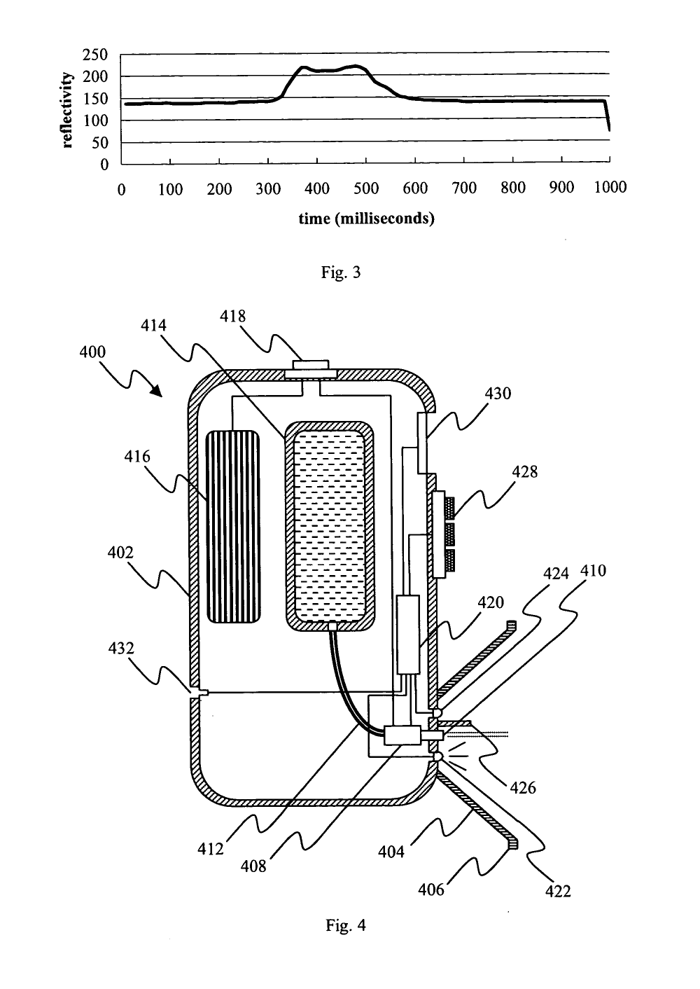

FIG. 3 is a graph of eye reflectivity of a subject sampled 100 times per second. This particular subject exhibits an open eye reflectivity having a scaled numerical value of about 140, and a closed eye reflectivity having a scaled numerical value of about 220. See Example 1 below for description of an apparatus that can provide such data. From FIG. 3 it will be clear that it is well within the capability of signal processing technology to distinguish between an open and a closed eye. A microprocessor can be programmed to quickly recognize the start and end of a blink based upon the scaled value of reflectivity, upon a change in that value, and/or upon the rate of change in that value.

The total duration of the blink recorded in FIG. 3 is about 250 milliseconds. During a first period of about 50 miliseconds, reflectivity increased from a low level to a high level, indicating closure of the eye. During a second period of about 120 milliseconds, reflectivity remained at the high level, indicating that the eye remained closed during that period. During a third period of about 80 milliseconds, reflectivity decreased to a low level similar to that prior to the first period, indicating re-opening of the eye.

A preferred apparatus of the invention for treating an eye automatically locks out operation of the applicator for the full duration of the blink, i.e., in the example shown in FIG. 3 from the beginning of the first period until the end of the third period.

A further illustrative apparatus of the invention, wherein the applicator is a bubble jet device, is shown schematically in FIG. 4.

The apparatus 400 of FIG. 4 comprises a hollow housing 402 having attached thereto a standoff, for example an eye-cup 404 with a rim 406 that is configured to engage a circumocular surface. A bubble jet device 408, disposed within the housing 22, has a nozzle 410 protruding through the housing at a location substantially in the center of the eye-cup 404 and oriented such that droplets issuing from the nozzle 410 are directed to an eye when the rim 406 of the eye-cup engages a circumocular surface. The bubble jet device 408 is fed via a conduit 412 from a refillable or replaceable reservoir 414 disposed within the housing 402 and accessible via an opening in the housing having a removable cover (not shown). The bubble jet device 408 is electrically energized by a battery 416 contained within the housing 402 and accessible via an opening in the housing having a removable cover (not shown). The battery 416 is electrically connected to the bubble jet device 408 via a circuit having an on/off switch, for example a push-button switch 418. A microprocessor 420 is conditioned to control the bubble jet device 408 such that volume, rate and/or spray pattern of the dispensed liquid can be varied. A light source, for example an LED 422, and a sensor 424 for measuring light reflected from an eye are located within the eye-cup 404 proximal to the nozzle 410. An opaque shield 426 prevents light from the LED 422 from impinging directly on the sensor 424. The sensor 424 and LED 422 are operatively connected to the microprocessor 420. A control interface, for example a touch pad 428, is provided for programming the microprocessor to operate the LED, sensor and bubble jet device in a desired fashion. An optional data display unit, for example a liquid crystal display unit 430, displays settings for the sensor and the bubble jet device and/or other information. Also optionally provided is an electronic interface 432 that enables connection of the microprocessor 420 to an external computer.

Ophthalmic diseases and disorders for diagnosis, prevention or treatment of which an eye treatment method of the invention can be useful include, without limitation, allergic diseases of the eye, for example allergic conjunctivitis, vernal keratoconjunctivitis and eyelid edema; dry eye; keratomalacia; trauma to the eye and adjacent tissues, including conjunctival and corneal foreign body injury, intraocular foreign body injury, contusion and laceration of eyelids, anterior chamber hemorrhage, and thermal and chemical burns of cornea, conjunctiva and eyelids; orbital cellulitis; chronic conjunctivitis; episcleritis; scleritis; superficial punctate keratitis; phlyctenular keratoconjunctivitis; interstitial keratitis; corneal ulcer, including peripheral ulcerative keratitis; uveitis, including iritis, cyclitis, choroiditis, retinitis and any combination thereof, and including uveitis caused by ankylosing spondylitis, Reiter's syndrome, juvenile rheumatoid arthritis, toxoplasmosis, cytomegalovirus, toxocariasis, histoplasmosis, sarcoidosis, tuberculosis and syphilis; Behcet's syndrome; sympathetic ophthalmnia; endophthalmitis; exophthalmos; bullous keratopathy; dacryostenosis; acute and chronic dacryocystitis; trichinosis; infective diseases of the eye, for example bacterial (e.g., staphylococcal) blepharitis of ulcerative and seborrheic types, bacterial and viral conjunctivitis (including trachoma and inclusion conjunctivitis), herpes simplex keratitis, and stye; acute retinal necrosis; chalazion; inversion and eversion of eyelids; neoplastic diseases including tumors of eyelids, intraocular tumor and malignant melanoma of choroid; cataract; cyctoid macular edema; birdshot choroidopathy; reticulum cell sarcoma; vascular retinopathies such as arteriosclerotic retinopathy and hypertensive retinopathy; diabetic retinopathy including non-proliferative and proliferative types; macular degeneration including atrophic and exudative types; retinal detachment; retinitis pigmentosa; glaucoma, including primary adult types (e.g., chronic open-angle glaucoma, acute and chronic angle-closure glaucomas, Posner-Schlossman syndrome), congenital (infantile) glaucoma, and secondary glaucoma resulting from pre-existing eye disease such as uveitis, intraocular tumor or cataract; papilledema; papillitis; retrobulbar neuritis; toxic amblyopia; optic atrophy; presbyopia; and ocular motility disorders including cranial nerve palsies.

Classes of ophthalmic drugs that can be delivered by the eye treatment method of the invention include, without limitation, demulcents; antimycotics, antibacterials, antivirals and other anti-infectives; steroids, NSAIDs, selective cyclooxygenase-2 inhibitors and other anti-inflammatory agents; acetylcholine blocking agents; adrenergic agonists, beta-adrenergic blocking agents, carbonic anhydrase inhibitors, prostaglandins and other antiglaucoma agents; antihypertensives; antihistamines; anticataract agents; and topical and regional anesthetics.

Illustrative specific drugs that can be delivered by the eye treatment method of the invention are acebutolol, aceclidine, acetylsalicylic acid (aspirin), N⁴ acetylsulfisoxazole, alclofenac, alprenolol, amfenac, amikacin, amiloride, aminocaproic acid, p-aminoclonidine, aminozolamide, anisindione, apafant, atenolol, azithromycin, bacitracin, benoxaprofen, benoxinate, benzofenac, bepafant, betamethasone, betaxolol, bethanechol, brimonidine, bromfenac, bromhexine, bucloxic acid, bupivacaine, butibufen, carbachol, carprofen, cefixime, cefoperazone, cefotaxime, ceftazidime, ceftizoxime, ceftriaxone, celecoxib, cephalexin, chloramphenicol, chlordiazepoxide, chlorprocaine, chlorpropamide, chlortetracycline, cicloprofen, cinmetacin, ciprofloxacin, clidanac, clindamycin, clonidine, clonixin, clopirac, cocaine, colistin, cromolyn, cyclopentolate, cyproheptadine, demecarium, dexamethasone, dibucaine, diclofenac, diflusinal, dipivefrin, domeclocycline, dorzolamide, doxycycline, enoxacin, epinephrine, erythromycin, eserine, estradiol, ethacrynic acid, etidocaine, etodolac, etoricoxib, fenbufen, fenclofenac, fenclorac, fenoprofen, fentiazac, flufenamic acid, flufenisal, flunoxaprofen, fluorocinolone, fluorometholone, flurbiprofen and esters thereof, fluticasone propionate, furaprofen, furobufen, furofenac, furosemide, gancyclovir, gentamicin, gramicidin, hexylcaine, homatropine, hydrocortisone, ibufenac, ibuprofen and esters thereof, idoxuridine, indomethacin, indoprofen, interferons, isobutylmethylxanthine, isofluorophate, isoproterenol, isoxepac, ketoprofen, ketorolac, labetolol, lactorolac, latanoprost, levo-bunolol, lidocaine, lonazolac, loteprednol, mafenide, meclofenamate, medrysone, mefenamic acid, mepivacaine, metaproterenol, methacycline, methanarnine, methylprednisolone, metiazinic, metoprolol, metronidazole, minocychne, minopafant, miroprofen, modipafant, nabumetome, nadolol, namoxyrate, naphazoline, naproxen and esters thereof, neomycin, nepafenac, nitroglycerin, norepinephrine, norfloxacin, nupafant, olfloxacin, olopatadine, oxaprozin, oxepinac, oxyphenbutazone, oxyprenolol, oxytetracycline, parecoxib, penicillins, perfloxacin, phenacetin, phenazopyridine, phenirarnine, phenylbutazone, phenylephrine, phenylpropanolamine, phospholine, pilocarpine, pindolol, pirazolac, piroxicam, pirprofen, polymyxin, polymyxin B, prednisolone, prilocaine, probenecid, procaine, proparacaine, protizinic acid, pyrimethamine, rimexolone, rofecoxib, salbutamol, scopolamine, silver sulfadiazine, sotalol, sulfacetamide, sulfanilic acid, sulfisoxazole, sulindac, suprofen, tenoxicam, terbutaline, tetracaine, tetracycline, theophyllarnine, timolol, tobramycin, tolmetin, travoprost, triamcinolone, trimethoprim, trospectomycin, unoprostone, valdecoxib, vancomycin, vidarabine, vitamin A, warfarin, zomepirac and pharmaceutically acceptable salts, esters and prodrugs thereof.

The eye treatment method of the invention is illustratively of particular utility in administration to an eye of one or more antiglaucoma agents, such as beta-adrenergic blocking agents, carbonic anhydrase inhibitors and prostaglandins, more particularly PGF2α derivatives. Illustrative beta-adrenergic blocking agents include betaxolol, timolol and salts thereof. Dorzolamide and salts thereof are illustrative carbonic anhydrase inhibitors. Illustrative PGF2α derivatives include latanoprost, travoprost and unoprostone. The eye treatment method is useful in administration of such a PGF2α derivative alone or in combination with one or more other drugs. In particular, combinations of a PGF2α derivative such as latanoprost with a beta-adrenergic blocking agent such as timolol can usefully be administered by the eye treatment method of the invention.

Such antiglaucoma agents are typically ocular hypotensive agents, effective in reducing intraocular pressure whether or not this is manifested as glaucoma. They can also be neuroprotective agents, stopping or retarding progressive damage to nerves resulting from glaucoma or other afflictions. Indications for such drugs, administered by the eye treatment method of the invention, therefore include, without limitation:
(a) ocular hypertension, including ocular hypertensive episodes following surgery or laser trabulectomy;
(b) congenital glaucoma
(c) open-angle glaucoma
(d) acute angle-closure glaucoma;
(e) chronic angle-closure glaucoma;
(f) secondary glaucoma arising from pre-existing ocular disease, for example inflammatory disease of the anterior segment, uveitis, intraocular tumor, enlarged cataract, central retinal vein occlusion, trauma, operative procedures or intraocular hemorrhage;
(g) retinal vascular diseases, including vasodilation of retinal and choroidal blood vessels;
(h) diabetic retinopathy; and
(i) non-glaucomatous ischemia.

Accordingly, in a preferred embodiment, the substance administered according to the eye treatment method of the invention is a composition comprising an antiglaucoma agent, for example a prostaglandin, illustratively latanoprost, in a dosage amount effective for treatment or prophylaxis of an ophthalmic disease or disorder selected from ocular hypertension, congenital glaucoma, open-angle glaucoma, acute angle-closure glaucoma, chronic angle-closure glaucoma, secondary glaucoma arising from pre-existing ocular disease, retinal vascular diseases, diabetic retinopathy and non-glaucomatous ischemia.

Drugs to be delivered by the present eye treatment method are first formulated as liquid compositions, that can, if desired, contain more than one drug. Liquid compositions include solutions, suspensions and solution/suspensions. It will be understood that the term “liquid” herein encompasses any flowable composition that can be applied by an applicator as herein contemplated. The drug is dissolved and/or suspended in a carrier liquid that is ophthalmically acceptable to form a composition useful in the eye treatment method of the invention.

### EXAMPLES

### Example 1

A computer controlled test apparatus was constructed. The test apparatus comprised a light source in the form of an LED capable of directing light toward a subject's eye and a sensor in the form of a receiver diode capable of receiving light reflected from the eye, and was controlled and powered by a microcontroller connected to a personal computer (PC).

Luminance of light from the LED reflected from an ocular surface was received by the sensor and converted into a voltage. The voltage provided an analog signal that was filtered to reduce background noise and passed through an amplifier before being routed to an analog/digital (A/D) converter in the microcontroller. The A/D converter transformed the voltage into a discrete numerical value between 0 and 255. This value was routed to the PC and displayed on the PC screen. To allow for calibration of the apparatus, five resistors in the amplifier were controlled via the microcontroller such that, by selectively actuating these resistors, gain could be adjusted.

The test apparatus had a 9V power supply with two voltage regulators to provide separate voltages for analog and digital portions of the apparatus, although some other power supply and distribution circuit could be used. The test apparatus had an eye-cup to standardize distance between the eye and the LED or sensor, with the LED and sensor spaced approximately 10 mm from the center of the pupil of the eye.

The test apparatus was constructed and deployed in five versions, each with a different LED, emitting light in a different spectrum: blue, green, yellow, red and infrared. The sensor was a receiving diode for visible light except in the version having an infrared LED, in which case the sensor was an infrared receiving diode. Adjustment of each apparatus was made to provide a high numerical value for reflectivity from a closed eye.

Eight subjects were selected, including two persons each with gray, green, blue and brown eyes. All five versions of the apparatus were tested on an eye of each subject. A series of 10 measurements were taken at a frequency of 100 Hz with the eye open, a further series of 10 measurements were taken at a frequency of 100 Hz with the eye closed, and a still further series of 100 measurements were taken at a frequency of 100 Hz. All subjects gave the same result: greater differences in reflectivity were exhibited with light in the red and infrared spectrum than with the other colors of light tested. Blue and green light gave the poorest results. This may have been due to poor sensitivity of the sensor to these colors of light. Eye color did not appear to affect the results, but differences in reflectivity appeared to be attributable to differences in size and shape of the eyes.

### Example 2

The apparatus of Example 1 having a red LED and visible light sensor, and the apparatus of Example 1 having an infrared LED and infrared sensor, were tested on the same 8 subjects as in Example 1. Measurements of reflectivity of open and closed eyes were made for each of the eight individuals on three successive days using both versions of the apparatus. Results with red light are presented in Table 1 and with infrared light in Table 2.

TABLE 1
Reflectivity with red light (scaled numerical value)

| Subject | Eye color | Eye state | Day 1 | Day 2 | Day 3 |
|---|---|---|---|---|---|
| 1 | brown | open | 91 | 98 | 102 |
| 1 | brown | closed | 135 | 148 | 120 |
| 2 | brown | open | 40 | 51 | 42 |
| 2 | brown | closed | 55 | 75 | 68 |
| 3 | green | open | 70 | 75 | 68 |
| 3 | green | closed | 155 | 182 | 157 |
| 4 | green | open | 92 | 84 | 98 |
| 4 | green | closed | 145 | 122 | 140 |
| 5 | blue | open | 78 | 100 | 92 |
| 5 | blue | closed | 118 | 113 | 99 |
| 6 | blue | open | 60 | 62 | 52 |
| 6 | blue | closed | 85 | 90 | 70 |
| 7 | gray | open | 70 | 74 | 67 |
| 7 | gray | closed | 104 | 110 | 102 |
| 8 | gray | open | 72 | 77 | 72 |
| 8 | gray | closed | 87 | 90 | 87 |

TABLE 2
Reflectivity with infrared light (scaled numerical value)

| Subject | Eye color | Eye state | Day 1 | Day 2 | Day 3 |
|---|---|---|---|---|---|
| 1 | brown | open | 170 | 200 | 170 |
| 1 | brown | closed | 205 | 215 | 210 |
| 2 | brown | open | 113 | 118 | 115 |
| 2 | brown | closed | 134 | 145 | 160 |
| 3 | green | open | 127 | 137 | 137 |
| 3 | green | closed | 185 | 230 | 212 |
| 4 | green | open | 147 | 150 | 120 |
| 4 | green | closed | 182 | 220 | 147 |
| 5 | blue | open | 143 | 120 | 138 |
| 5 | blue | closed | 185 | 160 | 155 |
| 6 | blue | open | 114 | 112 | 104 |
| 6 | blue | closed | 130 | 133 | 122 |
| 7 | gray | open | 120 | 118 | 120 |
| 7 | gray | closed | 154 | 154 | 150 |
| 8 | gray | open | 120 | 119 | 115 |
| 8 | gray | closed | 137 | 128 | 127 |

With both red and infrared light, the test apparatus measured a difference between an open eye and closed eye for each of the subjects.

As a result of further testing it was determined that positioning of the sensor is important, and that differences in reflectivity resulting from variation in positioning relative to the eye may be larger than differences due to the state of the eye. Thus it is desirable to construct devices based upon the principles of this invention that will minimize variation in positioning of the sensor relative to the eye. The standoff, for example, the eye-cup, provided in apparatus illustrated herein is therefore an important component. Even with a standoff, it may be desirable to calibrate the device each time it is used.

Testing also indicated that accuracy could be affected by shaking or moving the equipment. However, minor variations in measured intensity could be filtered out. Changes in reflectivity due to closing or opening of an eye can be readily differentiated from variations due to movement of the apparatus by the rapid change in reflectivity accompanying blinking as shown in FIG. 3.

It is contemplated that a focused LED might improve performance, but could make the apparatus more susceptible to variations in position of the apparatus. Sensors tuned to the particular LED wavelength emitted by the LED, shielding of the sensor from extraneous light, and shielding the eye from other light sources are also likely to improve performance. In absence of effective shielding from ambient light, it is preferred to use the apparatus in a dimly lit environment, or in light of wavelength to which the sensor is not sensitive.

What is claimed is:

1. A method for sensing the state of an eye of a subject, the method comprising measuring light reflected from an ocular surface and comparing the measured light to a reference.

2. The method of claim 1 wherein the subject is human.

3. The method of claim 1 that further comprises projecting light from a light source on to the ocular surface.

4. The method of claim 3 wherein the light source is a light emitting diode.

5. The method of claim 1 wherein the light measured is red or infrared light.

6. A method for treating an eye of a subject, the method comprising sensing the state of the eye by the method of claim 1, and controlling whether a substance is delivered to the eye whereby the substance is so delivered only when the eye is sensed to be open.

7. The method of claim 6 wherein the subject is human.

8. The method of claim 6 that further comprises projecting light from a light source on to the ocular surface.

9. The method of claim 8 wherein the light source is a light emitting diode.

10. The method of claim 6 wherein the light measured is red or infrared light.

11. The method of claim 6 wherein said sensing and controlling steps are configured to permit detection of a blink and lockout of delivery of the substance for at least the duration of the blink.

12. The method of claim 6 wherein light reflected from the ocular surface is measured with a sampling frequency of at least about 20 Hz.

13. The method of claim 6 wherein light reflected from the ocular surface is measured with a sampling frequency of at least about 50 Hz.

14. The method of claim 6 wherein light reflected from the ocular surface is measured with a sampling frequency of at least about 100 Hz.

15. The method of claim 6 wherein the substance delivered is useful for diagnosis, prevention or treatment of an ophthalmic condition or disorder selected from the group consisting of allergic diseases of the eye, dry eye, keratomalacia, trauma to the eye and adjacent tissues, orbital cellulitis, chronic conjunctivitis, episcleritis, scleritis, superficial punctate keratitis, phlyctenular keratoconjunctivitis, interstitial keratitis, corneal ulcer, uveitis, Behcet's syndrome, sympathetic ophthalmia, endophthalmitis, exophthalmos, bullous keratopathy, dacryostenosis, acute and chronic dacryocystitis, trichinosis, infective diseases of the eye, acute retinal necrosis, chalazion, inversion and eversion of eyelids, neoplastic diseases, cataract, cyctoid macular edema, birdshot choroidopathy, reticulum cell sarcoma, vascular retinopathies, diabetic retinopathy, macular degeneration, retinal detachment, retinitis pigmentosa, glaucoma, papilledema, papillitis, retrobulbar neuritis, toxic amblyopia, optic atrophy, presbyopia, and ocular motility disorders.

16. The method of claim 6 wherein the substance delivered is an ophthalmic drug selected from the group consisting of demulcents, antimycotics, antibacterials, antivirals, steroids, NSAIDs, selective cyclooxygenase-2 inhibitors, acetylcholine blocking agents, adrenergic agonists, beta-adrenergic blocking agents, carbonic anhydrase inhibitors, prostaglandins, antihypertensives, antihistamines, anticataract agents, and topical and regional anesthetics.

17. The method of claim 6 wherein the substance delivered is an ophthalmic drug selected from the group consisting of acebutolol, aceclidine, acetylsalicylic acid, N⁴ acetylsulfisoxazole, alclofenac, alprenolol, amfenac, amikacin, amiloride, aminocaproic acid, p-aminoclonidine, aminozolamide, anisindione, apafant, atenolol, azithromycin, bacitracin, benoxaprofen, benoxinate, benzofenac, bepafant, betamethasone, betaxolol, bethanechol, brimonidine, bromfenac, bromhexine, bucloxic acid, bupivacaine, butibufen, carbachol, carprofen, cefixime, cefoperazone, cefotaxime, ceftazidime, ceftizoxime, ceftriaxone, celecoxib, cephalexin, chloramphenicol, chlordiazepoxide, chlorprocaine, chlorpropamide, chlortetracycline, cicloprofen, cinmetacin, ciprofloxacin, clidanac, clindamycin, clonidine, clonixin, clopirac, cocaine, colistin, cromolyn, cyclopentolate, cyproheptadine, demecarium, dexamethasone, dibucaine, diclofenac, diflusinal, dipivefrin, domeclocycline, dorzolamide, doxycycline, enoxacin, epinephrine, erythromycin, eserine, estradiol, ethacrynic acid, etidocaine, etodolac, etoricoxib, fenbufen, fenclofenac, fenclorac, fenoprofen, fentiazac, flufenamic acid, flufenisal, flunoxaprofen, fluorocinolone, fluorometholone, flurbiprofen and esters thereof, fluticasone propionate, furaprofen, furobufen, fuirofenac, furosemide, gancyclovir, gentamnicin, gramicidin, hexylcaine, homatropine, hydrocortisone, ibufenac, ibuprofen and esters thereof, idoxuridine, indomethacin, indoprofen, interferons, isobutylmethylxanthine, isofluorophate, isoproterenol, isoxepac, ketoprofen, ketorolac, labetolol, lactorolac, latanoprost, levo-bunolol, lidocaine, lonazolac, loteprednol, mafenide, meclofenamate, medrysone, mefenamic acid, mepivacaine, metaproterenol, methacycline, methanamine, methylprednisolone, metiazinic, metoprolol, metronidazole, minocycline, minopafant, miroprofen, modipafant, nabumetome, nadolol, namoxyrate, naphazoline, naproxen and esters thereof, neomycin, nepafenac, nitroglycerin, norepinephrine, norfloxacin, nupafant, olfloxacin, olopatadine, oxaprozin, oxepinac, oxyphenbutazone, oxyprenolol, oxytetracycline, parecoxib, penicillins, perfloxacin, phenacetin, phenazopyridine, pheniramine, phenylbutazone, phenylephrine, phenylpropanolamine, phospholine, pilocarpine, pindolol, pirazolac, piroxicam, pirprofen, polymyxin, polymyxin B, prednisolone, prilocaine, probenecid, procaine, proparacaine, protizinic acid, pyrimetharnine, rimexolone, rofecoxib, salbutamol, scopolarnine, silver sulfadiazine, sotalol, sulfacetamide, sulfanilic acid, sulfisoxazole, sulindac, suprofen, tenoxicam, terbutaline, tetracaine, tetracycline, theophyllamine, timolol, tobramycin, tolmetin, travoprost, triamcinolone, trimethoprim, trospectomycin, unoprostone, valdecoxib, vancomycin, vidarabine, vitamnin A, warfarin, zomepirac and pharmaceutically acceptable salts, esters and prodrugs thereof.

18. The method of claim 6 wherein the substance delivered comprises at least one antiglaucoma agent.

19. The method of claim 18 wherein the at least one antiglaucoma agent is a beta-adrenergic blocking agent.

20. The method of claim 19 wherein the beta-adrenergic blocking agent is selected from the group consisting of betaxolol, timolol and salts thereof.

21. The method of claim 18 wherein the at least one antiglaucoma agent is a carbonic anhydrase inhibitor.

22. The method of claim 21 wherein the carbonic anhydrase inhibitor is dorzolamide or a salt thereof.

23. The method of claim 18 wherein the at least one antiglaucoma agent is a prostaglandin.

24. The method of claim 23 wherein the prostaglandin is a PGF2α derivative.

25. The method of claim 24 wherein the PGF2α derivative is selected from the group consisting of latanoprost, travoprost and unoprostone.

26. The method of claim 18 wherein the substance delivered comprises a PGF2α derivative in combination with at least one drug other than a PGF2α derivative.

27. The method of claim 26 wherein the at least one drug other than a PGF2α derivative is a beta-adrenergic blocking agent.

28. The method of claim 27 wherein the PGF2α derivative is latanoprost and the beta-adrenergic blocking agent is timolol.

29. The method of claim 18 wherein the substance is delivered in a dosage amount effective for treatment or prophylaxis of an ophthalmic disease or disorder selected from the group consisting of ocular hypertension, congenital glaucoma, open-angle glaucoma, acute angle-closure glaucoma, chronic angle-closure glaucoma, secondary glaucoma arising from pre-existing ocular disease, retinal vascular diseases, diabetic retinopathy, and non-glaucomatous ischemia.

30. A device for sensing the state of an eye, the device comprising a light source that directs light to an ocular surface of a subject, and a sensor for measuring light reflected from the ocular surface.

31. The device of claim 30 that further comprises a standoff to position and orient the sensor at a consistent distance from and angle to the eye.

32. The device of claim 31 wherein the standoff comprises an eye-cup having a distal rim adapted to contact a surface of the subject's face around the eye.

33. An apparatus for treating an eye of a subject, the apparatus comprising a device for sensing the state of an eye, an applicator for delivering a substance to the eye, and a control system that permits delivery of the substance when the sensing device detects that the eye is open but prevents delivery of the substance when the sensing device detects that the eye is closed; said sensing device comprising a light source that directs light to an ocular surface of a subject, and a sensor for measuring light reflected from the ocular surface.

34. The apparatus of claim 33 that further comprises a standoff to position and orient the sensor at a consistent distance from and angle to the eye.

35. The apparatus of claim 34 wherein the standoff comprises an eye-cup having a distal rim adapted to contact a surface of the subject's face around the eye.

36. The apparatus of claim 33 wherein said sensing device and said control system are configured to permit detection of a blink and lockout of delivery of the substance for at least the duration of the blink.
